(19) United States
(12) Patent Application Publication
(10) Pub. No.: US 2012/0041189 A1
(43) Pub. Date: Feb. 16, 2012

(54) PENTASACCHARIDE CRISTALLISE, SON PROCEDE D'OBTENTION ET SON UTILISATION POUR LA PREPARATION D'IDRAPARINUX

(75) Inventors: Philippe Clavel, Paris (FR); Xavier Lubeigt, Paris (FR); Pierre Potier, Paris (FR); Patrick Trouilleux, Paris (FR)

(73) Assignee: SANOFI, Paris (FR)

(21) Appl. No.: 13/130,501

(22) PCT Filed: Nov. 19, 2009
(86) PCT No.: PCT/FR2009/052221

§ 371 Date: Nov. 3, 2011

(30) Foreign Application Priority Data
Nov. 20, 2008 (FR) 0806491

(51) Int. Cl. C07H 1/00

(52) U.S. Cl. 536/123.12

(57) ABSTRACT

The Invention relates to the methyl pentasaccharide O-2,3,4-tri-O-methyl-α-D-glucopyranosyl-(1→4)-O-2,3-di-O-methyl-β-D-glucopyranosyluronic acid-(1→4)-O-α-D-glucopyranosyl-(1→4)-O-2,3-di-O-méthyl-α-L-idopyranosyluronic acid-(1→4)-O-α-D-glucopyranose in crystallised form, to a method for obtaining the same and to the use thereof for the preparation of idraparinux.

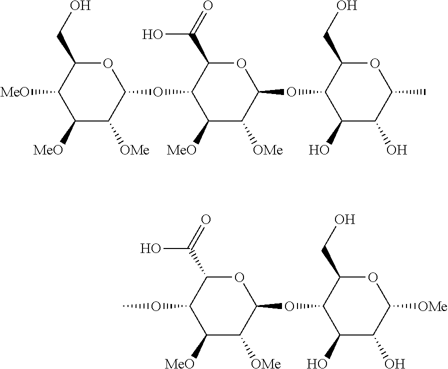

The present invention relates to a pentasaccharide, namely methyl O-2,3,4-tri-O-methyl-α-D-glucopyranosyl-(1→4)-O-2,3-di-O-methyl-β-D-glucopyranosyluronic acid-(1→4)-O-α-D-glucopyranosyl-(1→4)-O-2,3-di-O-methyl-α-L-idopyranosyluronic acid-(1→4)-O-α-D-glucopyranose, in crystalline form and to the process for obtaining it, and also to its use for the preparation of idraparinux.

Idraparinux sodium, or methyl O-2,3,4-tri-O-methyl-6-O-sodium sulfonato-α-D-glucopyranosyl-(1→4)-O-2,3-di-O-methyl-β-D-glucopyranosyluronate sodium-(1→4)-O-2,3,6-tri-O-sodium sulfonato-α-D-glucopyranosyl-(1→4)-O-2,3-di-O-methyl-α-L-idopyranosyluronate sodium-(1→4)-O-2,3,6-tri-O-sodium sulfonato-α-D-glucopyranose, is a pentasaccharide with antithrombotic activity.

The preparation of idraparinux by sulfatation of a deprotected pentasaccharide is described in Bioorganic & Medicinal Chemistry, 1994, Vol. 2, No. 11, pp. 1267-1280, and also in patent EP 0 529 715 B1.

A crystalline form of the pentasaccharide methyl O-2,3,4-tri-O-methyl-α-D-glucopyranosyl-(1→4)-O-2,3-di-O-methyl-β-D-glucopyranosyluronic acid-(1→4)-O-α-D-glucopyranosyl-(1→4)-O-2,3-di-O-methyl-α-L-idopyranosyluronic acid-(1→4)-O-α-D-glucopyranose has now been isolated. This compound in its crystalline form has proven to be very useful for the preparation of idraparinux, since it makes it possible to obtain this product in a particularly interesting chemical yield and with a significant gain in quality, the purity being improved as regards the crude product obtained, as will be detailed hereinbelow. These gains in reaction yield and in purity for the production of idraparinux are considerable advantages from an industrial viewpoint, since improving the robustness of a process is a constant cause for concern, especially in the case of large-scale syntheses.

One subject of the invention is thus the compound methyl O-2,3,4-tri-O-methyl-α-D-glucopyranosyl-(1→4)-O-2,3-di-O-methyl-β-D-glucopyranosyluronic acid-(1→4)-O-α-D-glucopyranosyl-(1→4)-O-2,3-di-O-methyl-α-L-idopyranosyluronic glucopyranose in crystalline form.

Methyl O-2,3,4-tri-O-methyl-α-D-glucopyranosyl-(1→4)-O-2,3-di-O-methyl-β-D-glucopyranosyluronic acid-(1→4)-O-α-D-glucopyranosyl-(1→4)-O-2,3-di-O-methyl-α-L-idopyranosyluronic acid-(1→4)-O-α-D-glucopyranose, referred to hereinbelow as the compound of formula (I), corresponds to the following formula:

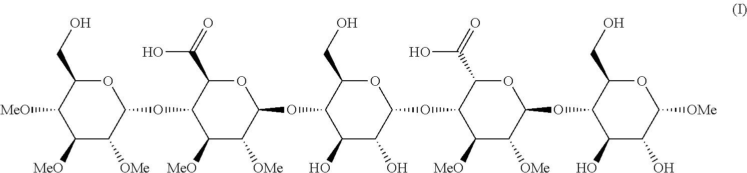

The compound of formula (I) in crystalline form according to the invention has a powder X-ray diffractogram whose characteristic lines are approximately at 12.009; 7.703; 7.300; 7.129; 5.838; 4.665; 4.476 and 3.785 angströms (interplanar distances). It also has a melting point of about 203° C. (203° C.±1° C.).

A subject of the invention is also a process for preparing the compound of formula (I) in crystalline form, which includes a step of crystallizing a compound of formula (I) in amorphous form in isopropanol or in an isopropanol/MTBE (methyl tert-butyl ether) mixture.

The compound of formula (I) may be obtained in crystalline form by dissolving the compound of formula (I) in amorphous form in hot isopropanol, followed by slow cooling of the reaction medium.

The term “dissolution in hot isopropanol” means isopropanol at a temperature that ensures the total dissolution of the compound of formula (I). Such a dissolution may be performed at a temperature of about 60-80° C., for example at 65-75° C.

The weight/volume ratio between the compound of formula (I) and isopropanol is advantageously about 1/6.

The term “slow cooling” means, under the known standard conditions of crystallization chemistry, a temperature ramp (rate) of about 10° C./hour, to arrive at a temperature of between, for example, 10 and 40° C., it being understood that the lower the temperature, the better the crystallization yield.

The crystallization of the compound of formula (I) may also be performed in the presence of a co-solvent, in particular in an isopropanol/MTBE mixture.

In this case, the isopropanol/MTBE mixture is advantageously a mixture of about 50/50 by volume. The process may especially be performed as follows:

1) dissolution of the compound of formula (I) in isopropanol, as described above,

2) cooling of the mixture to a temperature below the boiling point of the MTBE, followed by addition of MTBE, and

3) cooling of the mixture to a temperature of about 10° C.

During steps 1) and 2) above, the weight/volume ratios between the compound of formula (I) and, respectively, the isopropanol and the MTBE may each be about 1/6.

The cooling step 3) is preferably performed slowly for better control of the crystallization, and the mixture is advantageously maintained for one or more hours at a temperature of about 10° C., for example for about 2 hours.

The crystalline product may then be filtered off, washed and dried.

A compound of formula (I) in amorphous form may be obtained according to the teachings of the article Bioorganic & Medicinal Chemistry, 1994, Vol. 2, No. 11, pp. 1267-1280 and of patent EP 0 529 715 B1, namely, by total deprotection of a corresponding pentasaccharide bearing protective groups on the hydroxyl functions, such as acetyl and benzyl groups.

The compound of formula (I) may be obtained, for example, in amorphous form by hydrogenolysis of methyl O-2,3,4-tri-O-methyl-α-D-glucopyranosyl-(1→4)-O-2,3-di-O-methyl-β-D-glucopyranosyluronic acid-(1→4)-O-2-O-benzyl-α-D-glucopyranosyl-(1→4)-O-2,3-di-O-methyl-α-L-idopyranosyluronic glucopyranose, referred to hereinbelow as the compound of formula (I′), in which Me and Bn represent, respectively, methyl and benzyl:

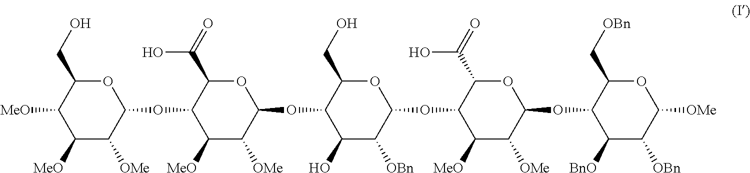

Such a hydrogenolysis may be performed in the presence of palladium-on-charcoal under hydrogen pressure, in a suitable solvent or solvent mixture, for example tetrahydrofuran, methanol, or a tetrahydrofuran/water, t-butanol/water or ethanol/water/ethyl acetate mixture.

The compound of formula (I′) may itself be obtained by saponification of methyl O-6-O-acetyl-2,3,4-tri-O-methyl-α-D-glucopyranosyl-(1→4)-O-benzyl-2,3-di-O-methyl-β-D-glucopyranosyluronate-(1→4)-O-3,6-di-O-acetyl-2-O-benzyl-α-D-glucopyranosyl-(1→4)-O-benzyl-2,3-di-O-methyl-α-L-idopyranosyluronate-(1→4)-O-2,3,6-tri-O-benzyl-α-D-glucopyranoside, referred to hereinbelow as the compound of formula (I″), in which Me, Bn and Ac represent, respectively, methyl, benzyl and acetyl groups:

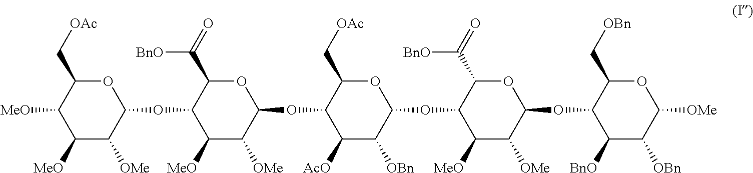

Such a saponification may be performed at a temperature between room temperature and 65° C. and advantageously between 25 and 55° C., using a base such as lithium, sodium or potassium hydroxide, and in a solvent such as tetrahydrofuran or acetonitrile.

The step of saponification of the compound of formula (I″) is advantageously followed by a step of precipitation of the saponified product. Such a precipitation may be performed in aqueous medium at a pH of about 1.5, for example using an aqueous hydrochloric acid solution. This significantly improves the purity of the saponified product obtained, as will be detailed hereinbelow.

A subject of the invention is also the use of the compound of formula (I) in crystalline form for the preparation of idraparinux.

More specifically, a subject of the invention is a process for preparing idraparinux by sulfatation of the compound of formula (I) in crystalline form as defined above.

The sulfatation may be performed using sulfur trioxide, for example in the form of a complex with triethylamine or pyridine, at a temperature between room temperature and about 50° C., for example between 20 and 50° C., in a solvent such as N,N′-dimethylformamide.

The step of sulfatation of the compound of formula (I) in crystalline form may advantageously be followed by a step of precipitation of the sulfated compound in a mixture of MTBE with one or two other solvents chosen from ethanol and isopropanol, for example in an MTBE/ethanol. MTBE/isopropanol or MTBE/ethanol/isopropanol mixture. Such a precipitation especially makes it possible to remove the solvent residues originating from the sulfatation.

Thus, a subject of the invention is a process for preparing idraparinux, which includes the following steps:

a) crystallization of a compound of formula (I) as defined previously, in amorphous form, in isopropanol, optionally in the presence of a co-solvent,

b) sulfatation of the compound of formula (I) in crystalline form obtained after the preceding step, to obtain idraparinux, and

c) optionally, precipitation of the idraparinux in a mixture of MTBE with one or two other solvents chosen from ethanol and isopropanol.

It has been seen previously that the compound of formula (I) in amorphous form may be obtained by hydrogenolysis of the compound of formula (I′). Thus, a subject of the invention is also a process for preparing idraparinux, which includes the following steps:

a₁) hydrogenolysis of a compound of formula (I′), as defined previously, to obtain a compound of formula (I) in amorphous form,

a) crystallization of the compound of formula (I) obtained after the preceding step in isopropanol, optionally in the presence of a co-solvent,

b) sulfatation of the compound of formula (I) in crystalline form obtained after the preceding step, to obtain idraparinux, and

c) optionally, precipitation of the idraparinux in a mixture of MTBE with one or two other solvents chosen from ethanol and isopropanol.

It has also been seen previously that the compound of formula (I′) may be obtained by saponification of the compound of formula (I″). Thus, a subject of the invention is also a process for preparing idraparinux, which includes the following steps:

a₃) saponification of a compound of formula (I″), as defined previously, to obtain a compound of formula (I′),

a₁) hydrogenolysis of the compound of formula (I′) obtained after the preceding step, to obtain a compound of formula (I) in amorphous form,

a) crystallization of the compound of formula (I) obtained after the preceding step in isopropanol, optionally in the presence of a co-solvent,

b) sulfatation of the compound of formula (I) in crystalline form obtained after the preceding step, to obtain idraparinux, and

c) optionally, precipitation of the idraparinux in a mixture of MTBE with one or two other solvents chosen from ethanol and isopropanol.

Finally, it has been seen previously that the saponification of the compound of formula (I″) may be followed by a step of precipitating the saponified product. Thus, a subject of the invention is also a process for preparing idraparinux, which includes the following steps:

a₃) saponification of a compound of formula (I″), as defined previously, to obtain a compound of formula (I′),

a₂) precipitation, in aqueous medium at a pH of about 1.5, of the compound of formula (I′) obtained after the preceding step,

a₁) hydrogenolysis of the compound of formula (I′) obtained after the preceding step, to obtain a compound of formula (I) in amorphous form,

a) crystallization of the compound of formula (I) obtained after the preceding step in isopropanol, optionally in the presence of a co-solvent,

b) sulfatation of the compound of formula (I) in crystalline form obtained after the preceding step, to obtain idraparinux, and

c) optionally, precipitation of the idraparinux in a mixture of MTBE with one or two other solvents chosen from ethanol and isopropanol.

In the processes described above, the crystallization step a) is advantageously performed in an isopropanol/MTBE mixture, as detailed previously.

The examples that follow illustrate in detail possible methods for obtaining the compound of formula (I) in crystalline form, the analytical characteristics of this crystalline form, and its use for preparing idraparinux.

### EXAMPLE 1

### Preparation of the Compound of Formula (I) in Crystalline Form (Scheme 1)

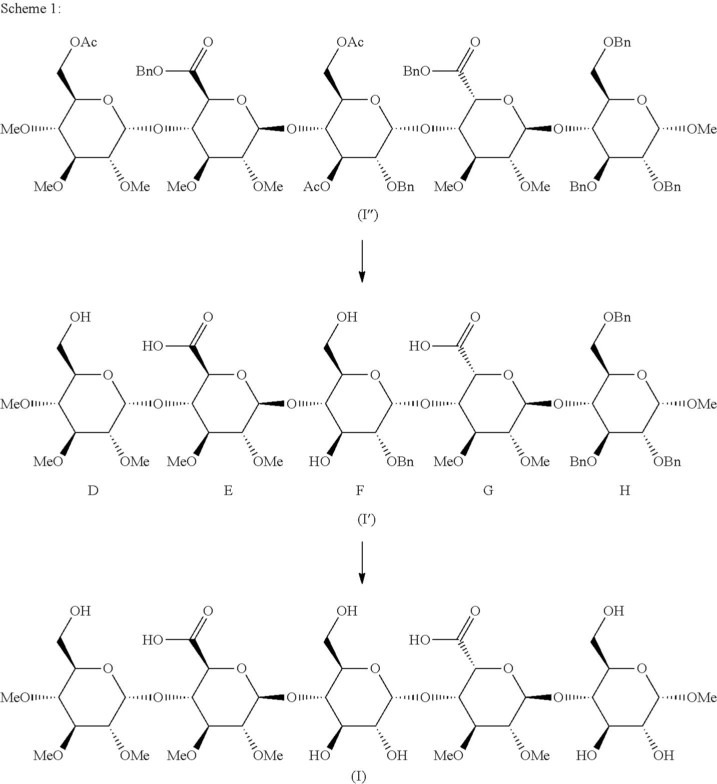

1.1: Preparation of the Compound of Formula (I′)

The compound of formula (I″) is obtained, for example, according to the teaching of patent EP 0 529 715 B1 or of the articles “Bioorg. Med. Chem.” (1994, Vol. 2, No. 11, pp. 1267-1280), “Bioorg. Med. Chem. Letters” (1992, Vol. 2, No. 9, pp. 905-910) or “Magnetic Resonance in Chemistry” (2001, Vol. 39, pp. 288-293). The compound of formula (I″) (5 g, 3.06 mmol) is dissolved in acetonitrile (10 mL). Deionized water (12.2 mL) and aqueous 30% sodium hydroxide solution (4.1 g) are then added. The mixture is heated to 40° C. and maintained at this temperature for 5 hours. The reaction medium is then cooled to 20° C. and acidified to pH 6.25 with aqueous 1N hydrochloric acid solution (about 17.7 g) before extraction with MTBE of certain impurities, the saponified product remaining in the aqueous phase. The residual acetonitrile, contained in the aqueous phase, is then removed by concentration, followed by diluting with deionized water (125 mL). The saponified product is finally precipitated at pH 1.5 by adding aqueous 1N hydrochloric acid solution (about 17.6 g) at 20° C. The suspension is maintained for 4 hours at 20° C. before filtration. The wet solid is finally dried in a vacuum oven at 30° C. to give 2.93 g (93.6%) of compound of formula (I).

NMR (anomeric protons of the saccharide units D, E, F, G, H): 5.79, 5.14, 5.55, 5.92, 4.94 ppm.

1.2 Preparation of the Crude Compound of Formula (I)

The compound of formula (I′) obtained after the preceding step is dissolved in tetrahydrofuran (18 mL). Palladium-on-charcoal (0.3 g) is added. The reaction medium is hydrogenated at 0.3 bar of hydrogen (relative pressure) for 4 hours. After filtering and evaporating, 2.12 g (99%) of the crude compound of formula (I) are obtained.

1.3: Preparation of the Compound of Formula (I) in Crystalline Form Using an Isopropanol/MTBE Mixture

The crude hydrogenated product obtained after the preceding step is dissolved in isopropanol (13 mL) at 65° C., and then crystallized at room temperature. The suspension is then cooled to 40° C., followed by addition of MTBE (13 mL), and is then cooled slowly to 10° C. After maintenance at 10° C. for 2 hours, the crystalline hydrogenated product is filtered off, washed and dried. 1.66 g of the compound of formula (I) in crystalline form are thus obtained, in the form of a cream-white powder. The reaction yield for the production of the compound of formula (I) in crystalline form, from the compound of formula (I′), is 92.5%. When expressed relative to the starting compound (I″), the reaction yield for the production of the compound of formula (I) in crystalline form is 86.6%.

NMR (anomeric protons of the saccharide units D, E, F, G, H) of the compound of formula (I) in crystalline form: 5.77, 5.11, 5.51, 5.84, 5.01 ppm.

1.4: Preparation of the Compound of Formula (I) in Crystalline Form Using Isopropanol

The crude hydrogenated product obtained after step 1.2 is dissolved in isopropanol (5 volumes) at 75° C. The medium is then cooled slowly until crystals appear, according to the known standard techniques for crystallization. The process is performed, for example, by a first step of cooling at 65° C. for 1 hour, and than a second step of cooling to a final temperature of 25° C. over 4 hours or of 5° C. over 6 hours, and finally maintenance at this final temperature for 30 minutes. The suspension is then filtered and rinsed with isopropanol (2×0.1 V) and compound (I) is isolated in the form of white crystals, which appear under a microscope in the form of needles. The ¹H NMR analysis of these crystals is identical to that described after step 1.3 above.

### EXAMPLE 2

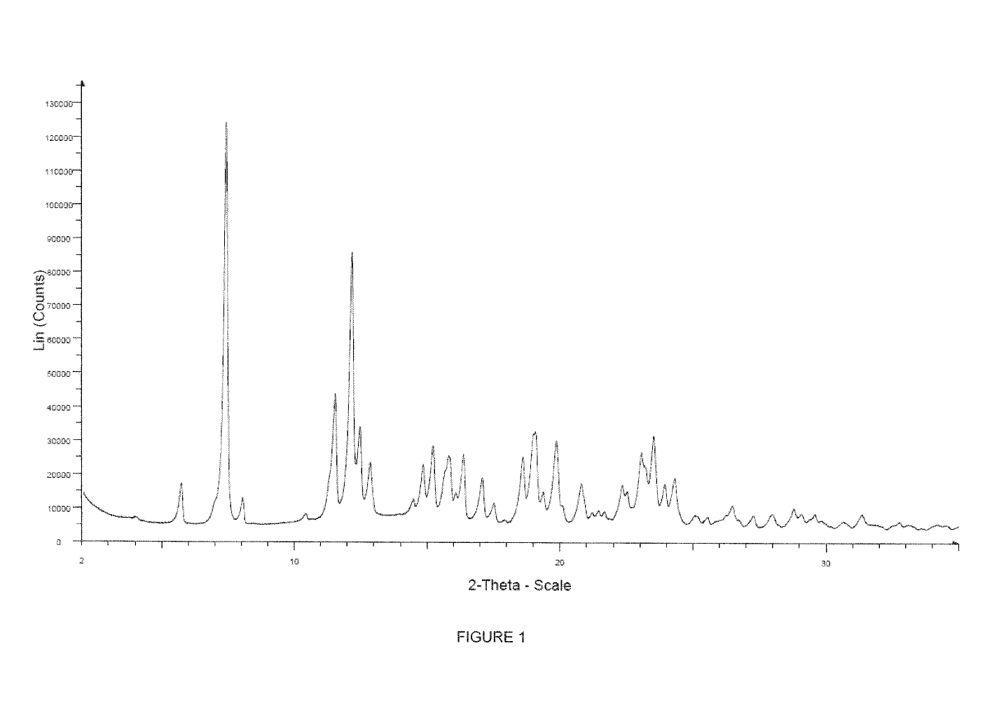

### Powder X-Ray Diffractogram of the Compound of Formula (I) in Crystalline Form (FIG. 1)

A D5005 machine (Brüker AXS) is used, under the following conditions:
support: flat sample holder,
angular domain: from 2.00 to 35.00°2θ (degrees 2-theta) in increments of 0.01°2θ,
time per increment: 70 seconds,
resolution slit: 0.1 mm,
generator: 50 kV and 40 mA.

The compound of formula (I) in crystalline form, as obtained in Example 1.3, is used as obtained and ground. FIG. 1 shows the X-ray diffractogram thus obtained. This diffractogram does indeed have the characteristic diffraction lines of a crystalline product.

A subject of the invention is thus a compound of formula (I) in crystalline form, characterized by the powder X-ray diffractogram according to FIG. 1 (mentioning the lines whose relative intensity is greater than or equal to 10%).

TABLE 1

| Angle 2-theta | Line (d, in | Intensity (counts | Relative |
|---|---|---|---|
| (°2θ) | angströms) | per second) | intensity (%) |
| 5.65 | 15.818 | 232.7 | 13.5 |
| 7.36 | 12.009 | 1725.2 | 100.0 |
| 7.97 | 11.084 | 174.6 | 10.1 |
| 11.48 | 7.703 | 618.9 | 35.9 |
| 12.11 | 7.300 | 1208.9 | 70.1 |
| 12.41 | 7.129 | 475.5 | 27.6 |
| 12.80 | 6.909 | 323.5 | 18.8 |
| 14.80 | 5.982 | 319.9 | 18.5 |
| 15.17 | 5.838 | 397.6 | 23.0 |
| 15.78 | 5.612 | 356.7 | 20.7 |
| 16.04 | 5.521 | 200.4 | 11.6 |
| 16.31 | 5.430 | 358.9 | 20.8 |
| 17.03 | 5.203 | 260.9 | 15.1 |
| 18.55 | 4.778 | 351.0 | 20.3 |
| 19.01 | 4.665 | 458.6 | 26.6 |
| 19.32 | 4.590 | 203.8 | 11.8 |
| 19.82 | 4.476 | 422.7 | 24.5 |
| 20.77 | 4.274 | 239.2 | 13.9 |
| 22.30 | 3.983 | 233.9 | 13.6 |
| 22.47 | 3.954 | 206.9 | 12.0 |
| 23.03 | 3.859 | 373.4 | 21.6 |
| 23.49 | 3.785 | 442.1 | 25.6 |
| 23.90 | 3.720 | 238.0 | 13.8 |
| 24.28 | 3.664 | 257.4 | 14.9 |

The determination of the lattice parameters of the crystalline structure of the compound of formula (I) was performed with the Reflex automatic indexation of the Materials Studio program followed by a Pawley refinement with the FullProff software. The crystalline system is the monocyclic network, the space group being P1 2₁ 1. The crystallographic data, namely the interplanar distances (a, b and c, in angströms), the angles (α, β and γ, in degrees) and the volume of each unit cell (V, in angströms³), are given in Table 2.

TABLE 2

| a (Å) | b (Å) | c (Å) | α (°) | β (°) | γ (°) | V (Å³) |
|---|---|---|---|---|---|---|
| 17.06 | 8.83 | 15.54 | 90.00 | 94.61 | 90.00 | 2333.5 |

### EXAMPLE 3

### Melting Point of the Compound of Formula (I) in Crystalline Form

An analysis by DSC (differential scanning calorimetry) of the compound of formula (I) in crystalline form, as obtained according to Example 1.3, is performed using a Perkin-Elmer “Pyris 1” machine, under the following conditions: from 25 to 250° C. at 3° C./minute, in a 30 μl aluminum crucible crimped with a pierced lid, under a stream of nitrogen. The analysis reveals an endotherm at 203° C.±1° C. (enthalpy: 66 J/g), corresponding to the melting point of the compound.

### EXAMPLE 4

### Preparation of Idraparinux from the Compound of Formula (I) in Crystalline Form (Scheme 2)

The preparation of idraparinux (II) from the compound of formula (I) is summarized in Scheme 2.

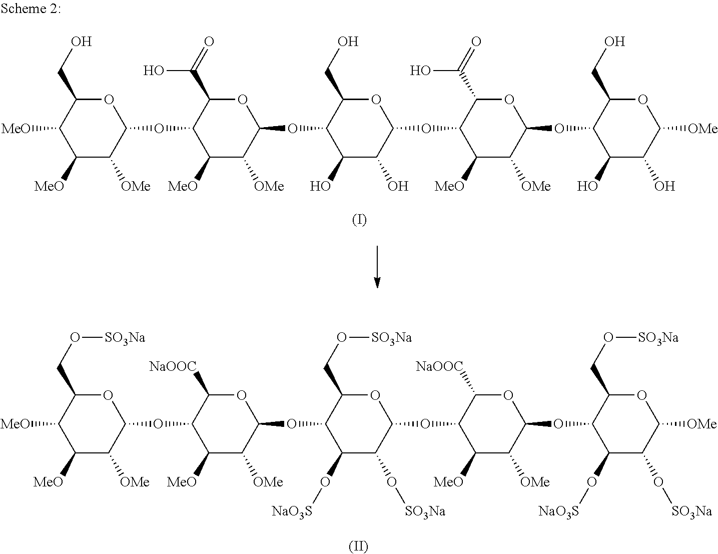

The compound of formula (I) in crystalline form, as obtained according to Example 1.3, is dissolved in N,N′-dimethylformamide (6.6 mL) and then heated to 30° C. Under an inert atmosphere, 3.8 g of pyridine-sulfur trioxide complex are added slowly, followed by maintenance at 30° C. for 4 hours. The reaction medium is then poured into aqueous 23.8% sodium hydrogen carbonate solution (16.3 g) maintained at a maximum of 25° C., to obtain the compound of formula (II). The reaction medium is kept stirring for hours. The solution of sulfated product is then poured onto an MTBE/isopropanol/ethanol mixture (171 mL/70 mL/70 mL). Precipitation of the product is observed, and, after filtering off, washing and drying the cake, 4.99 g (96.8%) of compound of formula (II) are obtained, and are then purified by anion-exchange chromatography according to the usual techniques.

NMR (anomeric protons of the saccharide units D, E, F, G, H) of the compound of formula (II): 5.48, 4.68, 5.44, 5.08, 5.18 ppm.

It thus appears that the process according to the invention makes it possible to obtain idraparinux (compound of formula (II)) in a chemical yield of about 84% (precisely 83.8% according to the protocols described above) starting from the compound of formula (I″), i.e. a gain in yield of about 30% relative to the process described in patent EP 0 529 715 B1.

### EXAMPLE 5

### Quality of The Synthetic Products and Intermediates According to the Process Performed in the Invention

The compound of formula (I) crystallized according to the present invention makes it possible to obtain idraparinux sodium not only in improved chemical yield, as detailed previously, but also with improved purity, which makes it possible to facilitate the final purification of the active principle. Specifically, an additional gain in yield, of about 5-10%, is observed as regards the final purification by anion-exchange chromatography. The use of a purer product in the subsequent chromatographic purification step and the ensuing improvement in chromatography yield are considerable industrial advantages.

Table 3 presents the levels of organic purity, and also, conversely, the levels of saccharide impurities, for the following compounds:
compound of formula (I′),
compound of formula (I), and
compound of formula (II) (idraparinux sodium),

said compounds being obtained either according to the teaching of patent EP 0 529 715 B1 (in which case the compound of formula (I) is in amorphous form), or according to the process according to the invention (in which case the compound of formula (I) is in crystalline form). The levels of these saccharide impurities are measured by HPLC.

TABLE 3

| | | Level (%) | |
|---|---|---|---|
| | | Production | Production |
| | | according to | according to |
| | | EP 0 529 715 B1 | (Schemes 1 and 2) |
| Compound (I′) | . purity | 75-85% | 90-92% |
| | . Σ impurities | 15-25% | 8-10% |
| Compound (I) | . purity | 75-85% | ≧95% |
| | . Σ impurities | 15-25% | ≦5% |
| Compound (II) | . purity | 75-82% | ≧95% |
| | . Σ impurities | 18-25% | ≦5% |

“Σ impurities” = sum of the impurities

It emerges from Table 3 that, firstly, the precipitation step for the production of the compound of formula (I′) and, secondly, the crystallization of the compound of formula (I) make it possible to obtain products with significantly improved organic purities.

In turn, the use of the crystalline product (I) in the saponification reaction (cf. Example 4) makes it possible to obtain idraparinux sodium in markedly higher quality than a product obtained from a non-crystalline compound of formula (I), as indicated by the organic purities presented in Table 3.

1. A compound methyl O-2,3,4-tri-O-methyl-α-D-glucopyranosyl-(1→4)-O-2,3-di-O-methyl-b-D-glucopyranosyluronic acid-(1→4)-O-α-D-glucopyranosyl-(1→4)-O-2,3-di-O-methyl-α-L-idopyranosyluronic acid-(1→4)-O-α-D-glucopyranose of formula (I): 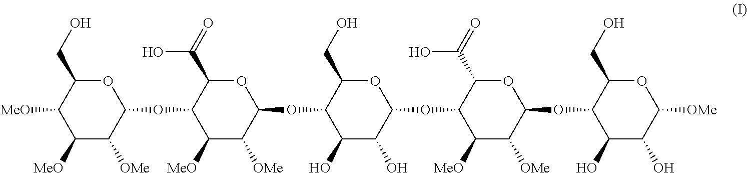
characterized in that it is in crystalline form.

2. The compound as claimed in claim 1, whose powder X-ray diffractogram presents the following characteristic lines, expressed as interplanar distances at approximately 12.009; 7.703; 7.300; 7.129; 5.838; 4.665; 4.476 and 3.785 angströms.

3. The compound as claimed in claim 1, characterized by the powder X-ray diffractogram according to FIG. 1.

4. The compound according to claim 1 having a melting point of 203° C.±1° C.

5. A process for preparing the compound of formula (I) comprising a step of crystallizing a compound of formula (I) in amorphous form in isopropanol, optionally in the presence of a co-solvent.

6. The process as claimed in claim 5, wherein the co-solvent is MTBE.

7. The process as claimed in claim 6, wherein the crystallization is performed in an isopropanol/MTBE mixture of about 50/50 by volume.

8. The process as claimed in claim 6, further comprising the following steps:
1) dissolution of the compound of formula (I) in isopropanol,
2) cooling of the mixture to a temperature below the boiling point of the MTBE, followed by addition of MTBE, and
3) cooling of the mixture to a temperature of about 10° C.

9. The process as claimed in claim 5, wherein the compound of formula (I) in amorphous form is obtained by hydrogenolysis of a compound of formula (I′):

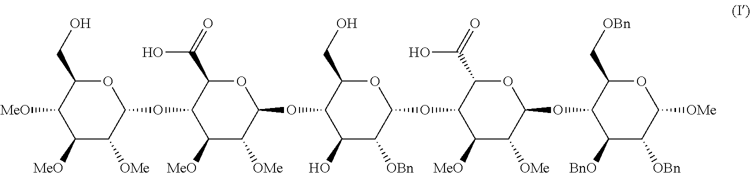

10. The process as claimed in claim 9, wherein the compound of formula (I′) is obtained by saponification of a compound of formula (I″):

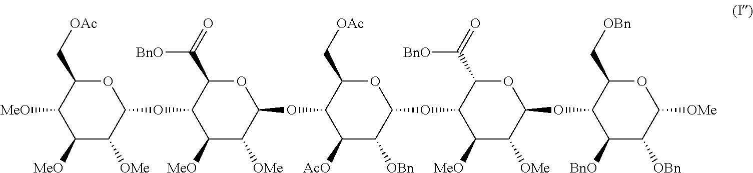

11. The process as claimed in claim 10, wherein the step of saponification of the compound of formula (I″) is followed by a precipitation in aqueous medium at a pH of 1.5.

12. A process for preparing idraparinux by sulfatation of the compound of formula (I) as claimed in claim 1.

13. The process as claimed in claim 12, wherein the step of sulfatation of the compound of formula (I) is followed by a step of precipitation in a mixture of MTBE with one or two other solvents chosen from ethanol and isopropanol.

14. The process as claimed in claim 13, wherein the precipitation step is performed in an MTBE/isopropanol/ethanol mixture.

15. The process as claimed in claim 11, including the following steps:
a) crystallization of a compound of formula (I), in amorphous form, in isopropanol, optionally in the presence of a co-solvent,
b) sulfatation of the compound of formula (I) in crystalline form obtained after the preceding step, to obtain idraparinux, and
c) optionally, precipitation of the idraparinux in a mixture of MTBE with one or two other solvents chosen from ethanol and isopropanol.

16. The process as claimed in claim 12, including the following steps:
a₁) hydrogenolysis of a compound of formula (I′), to obtain a compound of formula (I), in amorphous form,
a) crystallization of the compound of formula (I) obtained after the preceding step in isopropanol, optionally in the presence of a co-solvent,
b) sulfatation of the compound of formula (I) in crystalline form obtained after the preceding step, to obtain idraparinux, and
c) optionally, precipitation of the idraparinux in a mixture of MTBE with one or two other solvents chosen from ethanol and isopropanol.

17. The process as claimed in claim 12, including the following steps:
a₃) saponification of a compound of formula (I″), to obtain a compound of formula (I′),
a₁) hydrogenolysis of the compound of formula (I′) obtained after the preceding step, to obtain a compound of formula (I), in amorphous form,
a) crystallization of the compound of formula (I) obtained after the preceding step in isopropanol, optionally in the presence of a co-solvent,
b) sulfatation of the compound of formula (I) in crystalline form obtained after the preceding step, to obtain idraparinux, and
c) optionally, precipitation of the idraparinux in a mixture of MTBE with one or two other solvents chosen from ethanol and isopropanol.

18. The process as claimed in claim 12, including the following steps:
a₃) saponification of a compound of formula (I″), to obtain a compound of formula (I′),
a₂) precipitation, in aqueous medium at a pH of about 1.5, of the compound of formula (I′) obtained after the preceding step,
a₁) hydrogenolysis of the compound of formula (I′) obtained after the preceding step, to obtain a compound of formula (I), in amorphous form,
a) crystallization of the compound of formula (I) obtained after the preceding step in isopropanol, optionally in the presence of a co-solvent,
b) sulfatation of the compound of formula (I) in crystalline form obtained after the preceding step, to obtain idraparinux, and
c) optionally, precipitation of the idraparinux in a mixture of MTBE with one or two other solvents chosen from ethanol and isopropanol.

19. A process for preparing idraparinux comprising the steps of:
a) crystallization of a compound of formula (I), in amorphous form, in isopropanol, and
b) sulfatation of the compound of formula (I) in crystalline form obtained in step a.

20. The process according to claim 19, wherein the crystallization step is performed in the presence of a co-solvent.
